SEQUENCE LISTING (4 sequences)

SEQ ID NO: 1 - protein, 805 aa, Homo sapiens [sequence omitted]

SEQ ID NO: 2 - protein, 393 aa, Homo sapiens
QAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPK

SEQ ID NO: 3 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 4 - protein, 126 aa, Homo sapiens
KFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKK

(19) United States
(12) Patent Application Publication
(10) Pub. No.: US 2022/0074938 A1
(43) Pub. Date: Mar. 10, 2022

(54) METHOD OF DETECTING PATHOGENS AND/OR ANTIGENS IN SAMPLES

(71) Applicant: Senseutics Limited, London (GB)

(72) Inventors: Mona Kab OMIR, Rose Hill, Oxford (GB); David John LUNN, Abingdon, Oxfordshire (GB); Alexander James SHEPPARD, Manchester (GB)

(73) Assignee: Senseutics Limited, London (GB)

(21) Appl. No.: 17/314,983

(22) Filed: May 7, 2021

Related U.S. Application Data
(60) Provisional application No. 63/021,644, filed on May 7, 2020.

(30) Foreign Application Priority Data
May 7, 2021 (EP) PCT/EP2021/062222

(51) Int. Cl. G01N 33/569; G01N 33/543

(52) U.S. Cl. CPC G01N 33/56983; G01N 2333/165; G01N 33/54346; G01N 33/54388

(57) ABSTRACT

The present disclosure provides new and improved methods for the detection of pathogens, for example viral pathogens, in samples. The methods, tests and assays permit the identification of an infection, for example a Coronavirus infection, in a sample, but can also be used as a means to identify the infectious status or “infectivity level” of a subject. Disclosed is a method for detecting an antigen in a sample, said method comprising: contacting the sample with an antigen binding agent, wherein the antigen binding agent is not an antibody.

### RELATED APPLICATIONS

This application claims benefit and priority to PCT application PCT/EP2021/062222, which was filed on May 7, 2021. This application also claims priority to U.S. provisional application 63/021,644 filed on May 7, 2020. Both the PCT and the provisional application are incorporated herein in their entirety.

### FIELD

The disclosure provides tests, for example immunoassays, for use in detecting pathogens and/or antigens in samples, in particular, the disclosure provides point or care/point of need type tests.

### BACKGROUND

In January 2020, a novel coronavirus, severe acute respiratory syndrome coronavirus (SARS-CoV-2), was identified as the cause of an outbreak of viral pneumonia in Wuhan, China. The disease caused by SARS-CoV-2, called coronavirus disease (COVID-19), spread globally soon after. In the first three months after COVID-19 emerged nearly 3.6 million people were infected and over 250,000 died. In March 2020, the World Health Organization declared the COVID outbreak a pandemic.

The SARS-CoV-2 virus is considered to be easily transmissible, although researchers are still working to understand how it spreads. Data so far has shown that it spreads from person to person among those in close contact (within about 6 feet, or 2 meters). The virus spreads by respiratory droplets released when someone with the virus coughs, sneezes or talks.

These droplets can be inhaled or land in the mouth or nose of a person nearby. Therefore, efforts to date to contain the virus have focused on aggressive testing of individuals to find and isolate infected people.

Currently PCR, ELISA and antibody LFA tests are used to detect the presence of SARS-CoV-2. In the case of PCR, the virus is detected by amplifying the viral RNA and detecting this RNA, which necessitates multiple steps and specialised equipment in a lab setting. In the case of ELISAs, currently the virus is detected by the presence of proteins associated with the virus. However, ELISAs are inherently lab based as they require multiple steps and specialised equipment to bind and detect these proteins. Thus, neither PCR nor ELISA tests can be considered portable and administered rapidly at point of care/point of need. In the case of antibody LFAs, currently these devices only detect the antibodies which the human immune system generates in response to an infection by SARS-CoV-2. This means the infection can only be detected once there has been an immune response, which may be several days after the initial infection. This means existing LFA tests are of limited use when attempting to detect or diagnose current or active infections. Moreover, existing lateral flown tests, including those for the detection of covid-19 antigens, tend to rely on antibodies. Tests of this type can be vulnerable to a loss of sensitivity through mutations.

In view of these limitations, it would be advantageous to develop a rapid point of care, point of need, or home test kit which detects the presence of SARS-CoV-2 (and potentially other Coronavirus) in a test sample, to assist with the immediate detection or diagnosis of a current infection. The test kits described herein combine the benefits of the PCR or ELISA tests by directly detecting SARS-CoV-2, (without the need for specialised equipment) with the benefits of the traditional antibody LFAs which are POC but cannot detect the virus elements directly.

### SUMMARY

The present disclosure provides new and improved methods for the detection of pathogens, for example viral pathogens, in samples.

The methods, tests and assays described herein not only permit the identification of an infection (for example a Coronavirus infection) in a sample, but can also be used as a means to identify the infectious status or “infectivity level” of a subject to an extent that PCR and any other prior art assays cannot. For example, it is known that infected subjects can report as negative by DNA/RNA analysis, but positive for antigens. Clinically, in this scenario, the patient is therefore infectious, but perhaps asymptomatic with insufficient pathogen present to cause infection.

The methods described herein may find particular application in point of care or home test devices; these devices offer a means by which a sample, for example a biological fluid (including but not limited to blood, serum, plasma or saliva) may be rapidly and reliably tested for the presence of one or more pathogens and/or any component (for example an antigen) thereof.

One particular application of the methods described herein is in the detection of SARS-CoV-2 in a sample.

The methods described herein are rapid, reliable and sensitive. For example, a method or assay of this disclosure is capable of detecting as few as about 1×10² plaque forming units (PFU)/mL, 2×10² PFU/mL, about 5×10² PFU/mL, about 1×10³ PFU/mL, about 1.5×10³ PFU/mL, about 2×10³ PFU/mL, about 5×10³ PFU/mL or about 1×10⁴ PFU/mL.

It should be noted that the terms “comprise”, “comprising” and/or “comprises” is/are used to denote that aspects and embodiments of this disclosure “comprise” a particular feature or features. It should be understood that this/these terms may also encompass aspects and/or embodiments which “consist essentially of” or “consist of” the relevant feature or features.

A method for detecting Coronavirus or a Coronavirus antigen in a sample, said method comprising:
contacting the sample with a Coronavirus/Coronavirus antigen binding agent under conditions which permit binding between the binding agent and any Coronavirus or Coronavirus antigen present in the sample and detecting binding agent/Coronavirus complexes and/or binding agent/Coronavirus antigen complexes, wherein the Coronavirus/Coronavirus antigen binding agent is not an antibody.

A method for detecting Coronavirus or a Coronavirus antigen in a sample, said method comprising:
contacting the sample with a Coronavirus/Coronavirus antigen binding agent under conditions which permit binding between the binding agent and any Coronavirus or Coronavirus antigen present in the sample and detecting complexes comprising the binding agent and Coronavirus/Coronavirus antigen, wherein the Coronavirus/Coronavirus antigen binding agent is not an antibody.
It should be noted that the abovementioned Coronavirus/Coronavirus antigen binding agent may be one of a number of binding agents used in a method of this disclosure. Accordingly, the methods may use additional (for example second) Coronavirus/Coronavirus antigen binding agents. As noted, below, nothing about the terms first or second (as used herein) is intended to dictate the order in which these agents are used in the assays. Rather the terms are intended to distinguish one binding agent from another and it is the case that one (first) binding agent may be added before, concurrently with or after another (or second binding agent). Possible second bind agents are described elsewhere.

In a method of this disclosure, the detection of binding agent/Coronavirus complexes (i.e. complexes in which the binding agent has become bound to Coronavirus present in the sample) or binding agent/Coronavirus antigen complexes (i.e. complexes in which the binding agent has become bound to Coronavirus antigen present in the sample), might allow the user to conclude that the sample has been provided by or obtained from a subject that has or has been infected with Coronavirus.

The term Coronavirus as used herein should be understood as relating to those viruses which belong to the Coronaviridae family; these are a group of enveloped, positive-sense, single-stranded RNA viruses. Coronavirus causes respiratory tract infections in humans including for example, common cold type diseases and more severe acute respiratory syndromes. SARS-CoV, SARS-CoV-2 and MERS-CoV are examples of Coronaviruses that have emerged as pathogenic in humans. In humans, SARS-CoV is known to cause the disease known as severe acute respiratory syndrome (SARS) and SARS-CoV-2 causes the disease referred to as COVID-19.

The name “Coronaviridae” or “Coronavirus” is derived from the distinctive shape of the virus which contains a number of crown-like projections (“peplomers” or “spikes”). There are at least 4 structural proteins: the membrane protein (M), the envelope protein (E), the spike (glyco)protein (S) and the nucleocapsid (N) protein. The S-protein mediates host receptor binding, fusion and entry. The S-protein contains a number of domains or regions referred to as S1, S2 and or S-receptor binding domain (RBD). For convenience and throughout this specification, reference is made to the “S-protein”, this term should be understood as embracing not only the whole or complete S-protein, but also any host receptor binding fragment thereof and/or any S1 protein/domain, any S2 protein/domain and or the receptor binding domain of the S-protein (S-RBD). The term “S-protein” may also embrace pre-fusion forms of the S-protein, including trimeric forms. Without wishing to be bound by theory, the S-protein exists in a trimeric form on the virus with multiple s1, s2, and RBD subunits.

The S-protein from some Coronavirus (for example SARS-CoV and SARS-Cov-2) binds to the angiotensin converting enzyme 2 (ACE2) expressed on the surface of host cells.

The S-protein of MERS-CoV may bind dipeptidyl peptidase 4 (DPP4).

Any of the Coronavirus structural proteins may be referred to as Coronavirus antigens. For example, the S-protein may be referred to as a “Coronavirus antigen”.

In view of the above and in the context of this disclosure, wherever there is reference to Coronavirus or Coronavirus antigen, these terms should be understood as embracing any or all of the following:
(i) SARS-CoV-2
(ii) a SARS-CoV-2 antigen
(iii) the SARS-CoV-2 S-protein
(iv) the SARS-CoV-2 S-protein S1 domain
(v) the SARS-CoV-2 S-protein S2 domain
(vi) the SARS-CoV-2 S-protein RBD domain
(vii) SARS-CoV
(viii) a SARS-CoV antigen
(ix) the SARS-CoV S-protein
(x) MERS-CoV
(xi) MERS-CoV
(xii) MERS-CoV

In one teaching, the method of this disclosure may be a method for detecting the presence of the Coronavirus S-protein in a sample. It should be noted that any Coronavirus antigen (for example Coronavirus S-protein) may be present in a sample as free antigen or as antigen which is part of a whole or fragmented virus particle. In another teaching, any Coronavirus antigen (for example Coronavirus S-protein) may be present in a sample as a post-fusion, pre-fusion and/or trimeric form. An advantage of the methods and assays described herein is that it is possible to detect pre-fusion form of the Coronavirus S-antigen.

Additionally or alternatively, a method of this disclosure may provide a method for detecting, in a sample, one or more of:
(i) SARS-CoV;
(ii) a SARS-CoV antigen;
(iii) SARS-CoV S-protein;
(iv) SARS-CoV-2;
(v) SARS-CoV-2 antigen;
(vi) SARS-CoV-2 S-protein;
(vii) MERS-CoV;
(viii) MERS-CoV antigen; and/or
(ix) MERS-CoV S-protein

For example, a method for detecting SARS-CoV-2 and/or a SARS-CoV-2 antigen in a sample may comprise:
contacting the sample with a first SARS-CoV-2/SARS-CoV-2 antigen binding agent under conditions which permit binding between the binding agent and any SARS-CoV-2 or SARS-CoV-2 antigen present in the sample; and
detecting binding agent/SARS-CoV-2 complexes and/or binding agent/SARS-CoV-2 antigen complexes, wherein the first SARS-CoV-2/SARS-CoV-2 antigen binding agent is not an antibody.

In a method of this type, the detection of binding agent/SARS-CoV-2 complexes (i.e. a complex in which the binding agent has become bound to SARS-CoV-2 present in the sample) or binding agent/SARS-CoV-2 antigen complexes (i.e. complexes in which the binding agent has become bound to SARS-CoV-2 antigen present in the sample), might allow the user to conclude that the sample has been provided by or obtained from a subject that has or has been infected with SARS-CoV-2.

The methods of this disclosure may be used to diagnose Coronavirus infections and/or diseases in subjects. For example, the methods described herein may find application in the diagnosis of COVID-19, SARS and/or MERS—and a range of other Coronavirus related diseases.

The methods described herein may be used to detect the presence of Coronavirus and/or Coronavirus antigens in environmental samples including, for example samples of soil, dust, water, air, and/or on, within or from environmental swabs.

One of skill will appreciate that samples found to contain Coronavirus or Coronavirus antigens may have been provided by or obtained from subjects that have or have had a Coronavirus infection or a disease and/or condition associated therewith.

As described in more detail below, the methods of this disclosure may be adapted for use as point of care or point of need tests.

The sample may comprise blood (whole blood or a fragment thereof such as serum or plasma), a biopsy, cells, a tissue scraping, a mucosal or tissue secretion, mucus, mucus, saliva, pulmonary surfactant and fluid from a washing procedure (for example the fluid obtained from a lung or bronchoalveolar lavage washing procedure).

The sample may be provided by or obtained from any subject to be tested.

The subject may be any human or animal subject.

The subject may be suspected of having a Coronavirus infection and/or a disease or condition associated with or caused, or contributed to, by a Coronavirus.

The subject may have had or may be convalescing from a Coronavirus infection and/or a disease or condition associated with or caused, or contributed to, by a Coronavirus.

The subject may be asymptomatic and/or known to have been in contact with another (human or animal) subject that has or has had a Coronavirus infection and/or a disease or condition associated with or caused, or contributed to, by a Coronavirus.

The subject may have tested negative (by (RT)-PCR, ELISA or some other type of test) for a Coronavirus infection and/or Coronavirus associated disease or condition.

Diseases caused or contributed to by Coronavirus may include, for example, SARS, COVID-19 and/or MERS.

A first Coronavirus or Coronavirus antigen binding agent may comprise a moiety which binds to and/or has affinity and/or specificity for a Coronavirus antigen. For convenience, agents which have affinity and/or specificity for a particular antigen will be referred to as “binding to” that antigen. The term binding includes, but is not limited to, covalent binding, electrostatic binding, hydrogen bonding, hydrophobic binding and the like.

For example, the first Coronavirus or Coronavirus antigen binding agent may comprise a moiety which binds to the Coronavirus S-protein. The agent which binds a Coronavirus S-protein may bind to a fragment, domain or portion of the Coronavirus (S)-protein (for example the S1 domain, the S2 domain, S-RBD, or combinations thereof).

A method which is for the detection of SARS-CoV-2 in a sample may exploit an agent which binds a SARS-CoV-2 antigen, for example the SARS-CoV-2 S-protein. The agent which binds the SARS-CoV-2 antigen may bind to a fragment, domain or portion of the SARS-CoV-2 S-protein.

A method which is for the detection of SARS-CoV in a sample may exploit an agent which binds a SARS-CoV antigen, for example the SARS-CoV S-protein. The agent which binds the SARS-CoV antigen may bind to a fragment, domain or portion of the SARS-CoV S-protein.

A method which is for the detection of MERS-CoV in a sample may exploit an agent which binds a SARS-CoV antigen, for example the MERS-CoV S-protein. The agent which binds the MERS-CoV antigen may bind to a fragment, domain or portion of the MERS-CoV S-protein.

An agent for use in a method of this disclosure may comprise, for example, angiotensin-converting enzyme 2 (ACE2) or a Coronavirus or Coronavirus antigen binding fragment thereof. ACE2 may be used where the method is for the detection of SARS-CoV-2, a SARS-CoV-2 antigen, the SARS-CoV-2 S-protein, SARS-CoV, a SARS-CoV antigen or the SARS-CoV S-protein.

An agent for use in a method of this disclosure may comprise, for example, dipeptidyl peptidase 4 (DPP4) or a Coronavirus or Coronavirus antigen binding fragment thereof. DPP4 may be of particular use where the method is for the detection of MERS-CoV, a MERS-CoV antigen or the MERS-CoV S-protein.

For convenience, the term “ACE2” will embrace all ACE2 fragments which retain an ability to bind to Coronavirus and/or a Coronavirus antigen (in particular SARS-CoV-2, the SARS-CoV-2 S-protein, SARS-CoV or the SARS-CoV S-protein). For example, the term ACE2 will embrace fragments (for example truncated forms of ACE2) and/or dimeric/multimeric forms, which retain an ability to bind any Coronavirus S-protein and/or the S1 domain, S2 domain or S-RBD domain thereof.

Similarly and again, for convenience, the term “DPP4” will embrace all DPP4 fragments which retain an ability to bind to Coronavirus and/or a Coronavirus antigen (in particular MERS-CoV of the MERS-CoV S-protein).

Without wishing to be bound by theory, since some Coronavirus bind ACE2/DPP4, these proteins (ACE2 or DPP4) will, when brought into contact with a sample containing ACE2-binding or DPP4-binding Coronavirus, bind thereto and form a complex therewith. Those ACE2 or DPP4/Coronavirus and/or ACE2 or DPP4/Coronavirus antigen complexes can then be detected as used as means to confirm the presence of Coronavirus and/or Coronavirus antigen in the sample.

ACE2 may be used as an agent to bind SARS-CoV-2, SARS-CoV-2 S protein, SARS-CoV and/or SARS-CoV S protein, present in a sample. An advantage associated with the use of ACE2 as an agent to bind any SARS-CoV-2, SARS-CoV-2 S protein, SARS-CoV and/or SARS-CoV S protein, present in a sample is that the assay is not so vulnerable to a loss of sensitivity owing to a viral mutation.

DPP4 may be used as an agent to bind MERS-CoV or MERS-CoV S protein present in a sample.

Accordingly, in one teaching, the disclosure provides a method for detecting Coronavirus and/or a Coronavirus antigen in a sample may comprise:
contacting the sample with ACE2/DPP4 under conditions which permit binding between the ACE2/DPP4 and any Coronavirus or Coronavirus antigen present in the sample and detecting ACE2/DPP4:Coronavirus complexes and/or ACE2/DPP4::Coronavirus antigen complexes.

As stated and without wishing to be bound by theory, the presence of ACE2/DPP4::Coronavirus complexes (that is a complex comprising ACE2/DPP4 bound to a Coronavirus) and/or ACE2/DPP4::Coronavirus antigen complexes (a complex in which ACE2/DPP4 is bound to a Coronavirus antigen), may indicate that the test sample was provided by and/or obtained from a subject that has or has had a Coronavirus infection or a disease or condition caused or contributed to by a Coronavirus.

In another teaching, the invention provides a method for detecting SARS-CoV-2 and/or a SARS-CoV-2 antigen in a sample may comprise:
contacting the sample with ACE2 under conditions which permit binding between the ACE2 and any SARS-CoV-2 or SARS-CoV-2 antigen present in the sample and detecting ACE2/SARS-CoV-2 complexes and/or ACE2/SARS-CoV-2 antigen complexes.

Again, without wishing to be bound by theory, the presence of ACE2/SARS-CoV-2 complexes (that is a complex comprising ACE2 bound to a SARS-CoV-2) and/or ACE2/SARS-CoV-2 antigen complexes (a complex in which ACE2 is bound to a SARS-CoV-2 antigen), may indicate that the test sample was provided by and/or obtained from a subject that has or has had a SARS-CoV-2 infection or a disease or condition caused or contributed to by SARS-CoV-2 (for example COVID-19).

An exemplary (human) ACE2 sequence for use as a first SARS-CoV-2 binding agent in a method of this disclosure is deposited as Q9BYF1 (UniProtKB); that sequence is reproduced below as SEQ ID NO: 1

(human ACE2 sequence)
SEQ ID NO: 1

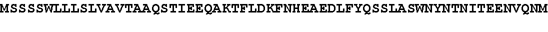

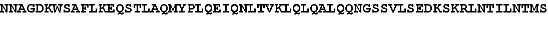

TIYSTGKVCNPDNPQECLLLEPGLNEIMANSLDYNERLWAWESWRSEVGKQLRPLYEEYVVL
KNEMARANHYEDYGDYWRGDYEVNGVDGYDYSRGQLIEDVEHTFEEIKPLYEHLHAYVRAKL
MNAYPSYISPIGCLPAHLLGDMWGRFWTNLYSLTVPFGQKPNIDVTDAMVDQAWDAQRIFKE

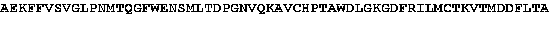

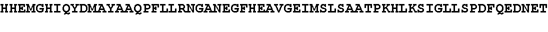

EINFLLKQALTIVGTLPFTYMLEKWRWMVFKGEIPKDQWMKKWWEMKREIVGVVEPVPHDET
YCDPASLFHVSNDYSFIRYYTRTLYQFQFQEALCQAAKHEGPLHKCDISNSTEAGQKLFNML
RLGKSEPWTLALENVVGAKNMNVRPLLNYFEPLFTWLKDQNKNSFVGWSTDWSPYADQSIKV
RISLKSALGDKAYEWNDNEMYLFRSSVAYAMRQYFLKVKNQMILFGEEDVRVANLKPRISFN
FFVTAPKNVSDIIPRTEVEKAIRMSRSRINDAFRLNDNSLEFLGIQPTLGPPNQPPVSIWLI
VFGVVMGVIVVGIVILIFTGIRDRKKKNKARSGENPYASIDISKGENNPGFQNTDDVQTSF

One or more of the residues marked in grey highlight are thought to be important in binding to SARS-CoV-2—in particular to the SARS-CoV-2 S-protein. These residues form a domain, all or part of which interacts with, binds to and/or has affinity for, the SARS-CoV-2 S-protein and/or the S-RBD part thereof (see below for a description of the S-RBD).

Thus the region spanning residues 24-416 of the ACE2 protein may referred to as defining or containing the SARS-CoV-2 binding interface.

This sequence is reproduced as SEQ ID NO: 2 below:

SEQ ID NO: 2

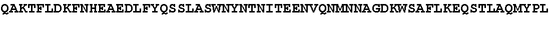

QEIQNLTVKLQLQALQQNGSSVLSEDKSKRLNTILNTMSTIYSTGKVCNPDNPQECLLLEPG
LNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMW

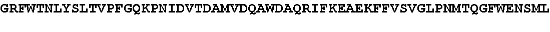

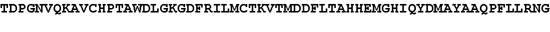

ANEGFHEAVGEIMSLSAATPK

In view of the above, a method of this disclosure may use, as a first Coronavirus (antigen) SARS-CoV-2 (antigen) binding agent, a peptide having or comprising a sequence of:
(a) SEQ ID NO: 1 or a SARS-CoV-2 binding fragment thereof; or
(b) SEQ ID NO: 2 or a SARS-CoV-2 binding fragment thereof.

For the avoidance of doubt, the term SARS-CoV-2 binding fragment, embraces any fragment of the ACE2 protein which functions to bind SARS-CoV-2 and/or the S-protein expressed thereby.

An exemplary (human) Dipeptidyl peptidase 4 (DPP4) sequence for use as a first binding agent in a method of this disclosure is deposited as P27487 (UniProtKB); that sequence is reproduced below as SEQ ID NO: 4

(human DPP4 sequence)
SEQ ID NO: 3
MKTPWKVLLG LLGAAALVTI ITVPVVLLNK GTDDATADSR
KTYTLTDYLK NTYRLKLYSL RWISDHEYLY KQENNILVFN
AEYGNSSVFL ENSTFDEFGH SINDYSISPD GQFILLEYNY
VKQWRHSYTA SYDIYDLNKR QLITEERIPN NTQWVTWSPV
GHKLAYVWNN DIYVKIEPNL PSYRITWTGK EDIIYNGITD
WVYEEEVFSA YSALWWSPNG TFLAYAQFND TEVPLIEYSF

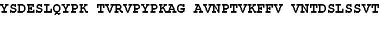

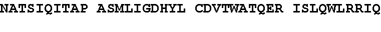

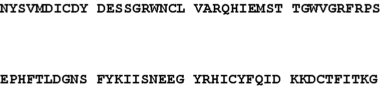

TWEVIGIEAL TSDYLYYISN EYKGMPGGRN LYKIQLSDYT
KVTCLSCELN PERCQYYSVS FSKEAKYYQL RCSGPGLPLY
TLHSSVNDKG LRVLEDNSAL DKMLQNVQMP SKKLDFIILN
ETKFWYQMIL PPHFDKSKKY PLLLDVYAGP CSQKADTVFR
LNWATYLAST ENIIVASFDG RGSGYQGDKI MHAINRRLGT
FEVEDQIEAA RQFSKMGFVD NKRIAIWGWS YGGYVTSMVL
GSGSGVFKCG IAVAPVSRWE YYDSVYTERY MGLPTPEDNL
DHYRNSTVMS RAENFKQVEY LLIHGTADDN VHFQQSAQIS
KALVDVGVDF QAMWYTDEDH GIASSTAHQH IYTHMSHFIK
QCFSLP

One or more of the residues marked in grey highlight are thought to be important in binding to Coronavirus—in particular to the MERS-CoV S-protein—but potentially also the S-proteins of other Coronavirus (including those described herein). These residues form a domain, all or part of which interacts with, binds to and/or has affinity for, the S-protein and/or the S-RBD part thereof.

Thus the region spanning residues 267-392 of the DPP4 protein may referred to as defining or containing a Coronavirus or Coronavirus S-protein SARS-CoV-2 binding interface.

This sequence is reproduced as SEQ ID NO: 4 below:

SEQ ID NO: 4
KFFVVNTDSL SSVTNATSIQ ITAPASMLIG DHYLCDVTWA
TQERISLQWL RRIQNYSVMD ICDYDESSGR WNCLVARQHI
EMSTTGWVGR FRPSEPHFTL DGNSFYKIIS NEEGYRHICY
FQIDKK

In view of the above, a method of this disclosure may use, as a first Coronavirus or Coronavirus antigen binding agent, a peptide having or comprising a sequence of:
(a) SEQ ID NO: 3 or a Coronavirus or MERS-CoV binding fragment thereof; or
(b) SEQ ID NO: 4 or a Coronavirus or MERS-CoV binding fragment thereof.

For the avoidance of doubt, the term Coronavirus or MERS-CoV binding fragment, embraces any fragment of the DPP4 protein which functions to bind Coronavirus or MERS-CoV and/or an antigen or S-protein expressed thereby.

In view of the above, in one teaching, a method for detecting a Coronavirus, a Coronavirus antigen, MERS-CoV or a MERS-CoV in a sample may comprise:
contacting the sample with an DPP4 protein or a Coronavirus or MERS-CoV binding fragment thereof, under conditions which permit binding between the DPP4 protein the Coronavirus or MERS-CoV binding fragment thereof and any Coronavirus, a Coronavirus antigen, MERS-CoV or MERS-CoV antigen present in the sample and detecting DPP4 protein or Coronavirus or MERS-CoV binding fragment thereof bound to Coronavirus, a Coronavirus antigen, MERS-CoV antigen or MERS-CoV.

The invention further embraces methods which use sequences which exhibit some degree of sequence identity and/or homology to any of the sequences described herein. For example the first SARS-CoV-2 binding agent may comprise a protein sequence which is at least 60%, at least 65%, at least 70%, at least 75%, at least 80%, at least 85%, at least 90%, at least 95%, at least 96%, at least 97%, at least 98%, at least 99% identical or homologous to the sequences represented by any of SEQ ID NOS: 1-4 described herein. Note, all useful homologous or identical sequences will be able to bind SARS-CoV-2 or at least the S-protein thereof.

It should be noted that all fragments or identical/homologous sequences can be tested for utility in the methods described herein by binding assays which test their binding affinity/specificity to Coronavirus and/or Coronavirus antigen(s).

In view of the above, in one teaching, a method for detecting SARS-CoV-2 in a sample may comprise:
contacting the sample with an ACE2 protein or a SARS-CoV-2 binding fragment thereof, under conditions which permit binding between the ACE2 protein or SARS-CoV-2 binding fragment thereof and any SARS-CoV-2 present in the sample and detecting ACE2 protein or SARS-CoV-2 binding fragment thereof bound to SARS-CoV-2.

The first Coronavirus/Coronavirus antigen binding agent of any of the methods described herein may be conjugated to (or bound to/associated with) some form of detectable particle, label or tag. In other words, the first Coronavirus/Coronavirus antigen binding agent for use in any of the methods described herein may be labelled or tagged for detection.

The first binding agent may comprise an optically detectable label or tag.

The first binding agent may comprise a fluorescent or chemiluminescent label or tag.

The first binding agent may be conjugated to biotin (i.e. it may be biotinylated).

The first binding agent may be fused or conjugated to a peptide tag. Suitable peptide tags may include, for example peptide tags which comprise one or more amino acids. For example, a suitable peptide tag may comprise multiple histidine amino acids—a ‘His-tag’.

The first binding agent may comprise a nanoparticle. Suitable nanoparticles may include any to which a first binding agent of this disclosure can be covalently or passively bound and/or immobilised, for example, to the surface.

A nanoparticle for use may comprise, consist of or consist essentially of a gold nanoparticle and/or a gold-coated nanoparticle (for example a gold coated silica nanoparticle).

The nanoparticle may not comprise a gold nanoparticle. For example, the nanoparticle may comprise a non-gold nanoparticle. One of skill will appreciate that any nanoparticle/visualisation agent which has been used in a lateral flow assay can be applied to the methods described herein. For example, the nanoparticle may comprise any (inorganic) element which has found application as a nanoparticle/visualisation agent in assays of the type described herein

The first binding agent may comprise a coloured latex beads.

The size of the nanoparticle may vary depending on a number of factors. Again, without wishing to be bound to any theory, larger particle may allow more target binding and may be easier to visualise.

Particles of between 50 nm and 500 nm in diameter may be useful. Nanoparticles with a diameter of about 100 nm, about 200 nm, about 300 nm, or about 400 nm may be used.

Nanoparticles with a diameter of >100 nm, >200 nm and/or >300 nm may be used. The nanoparticles may each be the same or of different sizes. For example, larger particles may be capable of being bound to more of the first binding agent (there is more surface area to which the first binding agent may be bound and/or immobilised). Without wishing to be bound by theory, it is suggested that by increasing the amount of first biding agent available to bind Coronavirus and/or Coronavirus antigen in a sample, it is possible to improve the sensitivity of an assay of this disclosure.

Accordingly, a method of this disclosure may use:
(i) a conjugated SARS-CoV-2 binding agent;
(ii) a conjugated SARS-CoV binding agent; or
(iii) a conjugated MERS-CoV binding agent.

As stated, any of binding agents (i)-(iii) above may bind a SARS-CoV-2 antigen, a SARS-CoV antigen or MERS-CoV antigen and may be either covalently or passively conjugated to a gold nanoparticle or some other detectable (for example optically detectable) tag. The first binding agent may be immobilised to or impregnated into/held within a substrate.

For example, the first binding agent may be held within or impregnated into a nitrocellulose membrane. This may be impregnated through the use of a tag (for example, biotin).

When solubilised, the first agent may mobilise through the substrate (for example the nitrocellulose membrane). Solubilisation may occur upon the addition of a sample.

A method of this disclosure may comprise an optional wash step. For example, once prior to any detection step, the method may be subject to a wash so as to remove any sample from the system. The wash may also remove any material which has not bound to the first binding agent. A wash may use a wash buffer. For example a wash may use a drying buffer. A suitable drying buffer may comprise (5%) Sucrose, (3%) BSA, (0.5%) Tween 20, (0.1%) sodium azide and (10 mM) Borate buffer. The pH of this buffer may be above about 6.9. Without wishing to be bound by theory, the presence of sucrose in this buffer may slow down the travel sample through the membrane in the washing step. Other wash buffers may comprise, for example, phosphate buffered saline (PBS).

The methods described herein may use a second agent, which second agent also binds the Coronavirus or Coronavirus antigen. It should be noted that the terms “first” and “second” as used herein do not necessarily refer to the order in which the binding agents are added to, or contacted with, the sample. Rather, the terms “first” and “second” primarily refer to the fact that the methods and assays of this disclosure may use two agents capable of binding a Coronavirus antigen (in other words, each binding agent is one of at least two used in any of methods or assays described herein)—either one or those agents may be added to or contacted with the sample, before, concurrently with, or after, the other. Accordingly in one teaching, any of the disclosed methods or assays may involve contacting a sample with what is described herein as the “first coronavirus binding agent” to form (if the sample contains Coronavirus/Coronavirus antigen) first coronavirus binding agent::Coronavirus/Coronavirus antigen complexes and then contacting the sample (or any first coronavirus binding agent::Coronavirus/Coronavirus antigen complexes) with what is described herein as the second (or additional) coronavirus binding agent. In another teaching, the disclosed methods or assays may involve contacting a sample with what is described herein as the “second coronavirus binding agent” to form (if the sample contains Coronavirus/Coronavirus antigen) second coronavirus binding agent::Coronavirus/Coronavirus antigen complexes and then contacting the sample (or any second coronavirus binding agent::Coronavirus/Coronavirus antigen complexes) with what is described herein as the first (or additional) coronavirus binding agent. In a further teaching, any of the disclosed methods or assays may involve contacting a sample with both the “first Coronavirus binding agent” and the “second Coronavirus binding agent. Following the addition of both binding agents, the method will (if the sample contains Coronavirus/Coronavirus antigen) complexes which comprise not only the first and second Coronavirus binding agents but also the Coronavirus and/or Coronavirus antigen. Accordingly, in one teaching, a method for detecting Coronavirus or a Coronavirus antigen in a sample, said method comprising:
contacting the sample with a first Coronavirus/Coronavirus antigen binding agent under conditions which permit binding between the binding agent and any Coronavirus or Coronavirus antigen present in the sample to form a first agent/Coronavirus or a first agent/Coronavirus antigen, complex;
contacting (the sample with) a second Coronavirus/Coronavirus antigen binding agent under conditions which permit binding between any complexes and the second binding agent;
detecting first agent/Coronavirus/second agent complexes (that is a complex in which both the first and second agents are bound to a Coronavirus) and/or first agent/Coronavirus antigen/second agent complexes (that is a complex in which both the first and second agents are bound to a Coronavirus antigen), which indicates that the sample may have been provided by, or obtained from, a subject that has, or has had, a Coronavirus infection and/or a disease or condition associated therewith (for example COVID-19).
As stated, it should be understood that the first binding agent may be contacted with the sample, before, concurrently with, or after the second binding agent.

A method which includes the use of first and second binding agents may include an optional washing step. For example, the method may include at least two washing steps. A first wash step may be executed after addition of the sample to the first binding agent and before addition of the second binding agent. The second optional washing step may be executed after addition of the second binding agent and before the detecting step.

The second binding agent may comprise any molecule which binds to a Coronavirus and/or a Coronavirus antigen—including any or all of the Coronavirus types described herein (e.g. SARS-CoV-2, SARS-CoV and MERS-CoV) or any or all of the Coronavirus antigens described herein, for example the Coronavirus S-protein, the SARS-CoV-2 S-protein, the SARS-CoV S-protein or the MERS-CoV S-protein).

The second agent may be the same as the first binding agent. For example, the second binding agent may also comprise ACE2 and/or DPP4 (where the terms “ACE2” and “DPP4” embrace all Coronavirus or Coronavirus antigen binding fragments of ACE2 and/or DPP4 as defined herein).

The second agent may comprise an antibody.

The second agent may comprise an antibody which binds to (i.e. has affinity and/or specificity for) a Coronavirus and/or a Coronavirus antigen.

The second agent may bind the same target as the first binding agent. For example, the second agent may bind a different site on the same target as the first binding agent.

The second agent may bind the S2 domain of the Coronavirus S-protein. In one teaching, the second agent may comprise an anti-S2 antibody (that is an antibody with specificity for the S2 domain of the SARS-CoV-2 Spike protein S2 domain.

The second agent may be conjugated. For example the second agent may be conjugated to a detectable moiety.

The second agent may be conjugated or bound to a detectable moiety, for example an optically detectable moiety (a gold coated nanoparticle or the like). The second agent may be conjugated to, for example, europium nanoparticles and/or carbon nanoparticles.

The second agent may be biotinylated (a biotinylated agent may bind to the PSA that is immobilised on the surface of a substrate, for example a nitrocellulose membrane).

The second agent may comprise an antibody.

The second agent may comprise a biotinylated antibody.

The second agent may comprise a biotinylated antibody which binds to Coronavirus and/or a Coronavirus antigen.

The second binding agent may be immobilised to or impregnated into/held within a substrate.

For example, the second binding agent may be held within or impregnated into a nitrocellulose membrane.

When solubilised, the second agent may mobilise through the substrate (for example the nitrocellulose membrane). Solubilisation may occur upon the addition of a sample.

Where the method is for the detection of SARS-CoV-2 and/or a SARS-CoV-2 antigen (for example the SARS-CoV-2 S-protein) in a sample, the second agent may comprise an antibody which binds SARS-CoV-2 and/or a SARS-CoV-2 antigen.

Where the method is for the detection of SARS-CoV and/or a SARS-CoV antigen (for example the SARS-CoV S-protein) in a sample, the second agent may comprise an antibody which binds SARS-CoV and/or a SARS-CoV antigen.

Where the method is for the detection of MERS-CoV and/or a MERS-CoV antigen (for example the MERS-CoV S-protein) in a sample, the second agent may comprise an antibody which binds MERS-CoV-2 and/or a MERS-CoV antigen.

In all cases, a method of this disclosure may involve a control test or assay in which the results obtained from a test sample are compared with the results obtained from positive and/or negative control samples. For example, a negative control sample may comprise a sample known to lack Coronavirus and/or Coronavirus antigens. For example, where a method is used to determine whether or not a subject has a Coronavirus, a SARS, a SARS-CoV-2 or a MERS-CoV infection or an associated disease, the negative control may exploit a sample which does not Coronavirus, SARS, SARS-CoV-2 or MERS-CoV or any antigens therefrom.

A positive control sample may comprise a sample which contains (or has been spiked with) a Coronavirus, SARS, SARS-CoV-2, MERS-CoV or any antigen therefrom. In assays such as, for example, lateral flow assays, a positive control may function simply to show that a test has run and functioned correctly. As such, a positive control may comprise an antibody that binds to a component expected to be present in the test sample. For example, where the sample is a saliva, the positive control may comprise an antibody which binds to salivary amylase or the like.

The test sample and the control sample may be the same. For example, where the test sample is, or comprises blood (or a fragment thereof such as serum or plasma), so too is, or does, the control sample.

The disclosure provides a method wherein the first and second binding agents are added to a sample to be tested. The sample (with the added first and second binding agents) may then be incubated for a period of time. For example the period of incubation may last seconds, minutes or hours. The period of time may be 5 s, 10 s, 15 s, 20 s, 30 s, 60 s, 2 min, 5 min, 10 min, 15 min, 20 min, 30 min, 40 min, 50, min, 1 hr, 2 hr or 3 hr. Without wishing to be limited a period of 10 s may be useful in the case of a sample comprising S-RBD and S1+S2 domain and a time period of about 15 min may be useful in the case of a sample that comprises the S1 protein domain). Whatever the time period, it may be suitable to allow binding between the first and second agents and any Coronavirus or Coronavirus antigen present in the sample. The sample (with the added first and second binding agents) may then be added to a system which comprises a capture agent, wherein the capture agent binds to the biotinylated second agent. It should be noted that in a method of this type, either the first or second binding agent may be biotinylated. Additionally, the capture agent may comprise polystrepavidin (PSA). The capture agent may be immobilised at or on a test line in substrate. The sample (with added first and second binding agents which may have bound to any Coronavirus or Coronavirus antigen present in the sample), may then be added to the substrate and allowed or caused to flow there through. For example the substrate may comprise an absorbent pad that draws the sample through the substrate. In this teaching, the first and/or second binding agents may comprise either ACE2 or DPP4.

Any of the methods described herein may be conducted as part of an immunoassay.

Accordingly, the disclosure further provides an immunoassay for the detection of Coronavirus in a sample, wherein the immunoassay comprises a labelled agent which binds a Coronavirus and/or a Coronavirus, wherein the labelled agent does not comprise an antibody.

The immunoassay may comprise a lateral flow assay.

A lateral flow assay may comprise a sample pad (for receiving the sample), a test line, a control line and an absorbent pad (to draw the sample through the assay).

A lateral flow assay of this disclosure may be for the detection of Coronavirus, a Coronavirus antigen, SARS-CoV-2, SARS-CoV, MERS-CoV, the S-protein of SARS-CoV-2, the S-protein of SARS-CoV or the S-protein of MERS-CoV.

A lateral flow assay of this disclosure may comprise the first and second binding agents described herein.

For example, the lateral flow assay may contain an ACE2 protein (again for the avoidance of doubt, the term “ACE2” includes proteins having the sequences defined herein and also any functional fragments thereof). The lateral flow assay may comprise a nanoparticle coated with or bound to multiple copies of the ACE2 protein. As stated, useful nanoparticles may comprise inorganic elements and/or particles containing organic materials, including, but not limited to, gold.

The lateral flow assay may contain a DPP4 protein (again for the avoidance of doubt, the term “DPP4” includes proteins having the sequences defined herein and also any functional fragments thereof). The lateral flow assay may comprise a nanoparticle passively conjugated with or covalently bound to multiple copies of the DPP4 protein. As stated, useful nanoparticles may comprise inorganic elements, including, but not limited to, gold.

The lateral flow assay may further comprise an antibody which binds the same target as the ACE2 protein. For example, the antibody may also bind the Coronavirus S-protein. An antibody for use in a lateral flow assay of this innovation may comprise an anti-S2 antibody—that is an antibody with specificity/affinity for the S2 domain of the SARS-Cov-2 spike protein.

In view of the above, a lateral flow assay of this disclosure may comprise a nanoparticle comprising multiple copies of an ACE2 protein (as defined herein) and an anti-S2 antibody. Wherein a sample is brought into contact with the ACE2 conjugated nanoparticle under conditions which permit binding between the ACE2 component of the nanoparticle and any Coronavirus/Coronavirus S-protein present in the sample. Where the sample contains a quantity of Coronavirus/Coronavirus S-protein, a nanoparticle/S-protein complex will form via binding between the ACE2 component of the nanoparticle and the S-protein present in the sample (present in any pre-fusion, trimeric or post-fusion form or as S-protein expressed on the surface of a Coronavirus particle). The nanoparticle/S-protein complex may then mobilise through the assay whereupon it will contact the second binding agent. As sated, the second binding agent will also bind the Coronavirus/Coronavirus S-protein component of the nanoparticle/S-protein complex. The second binding agent may bind a different site on the S-protein and may comprise a label or tag (referred to hereinafter as a ‘capture moiety’)—for example, the second binding agent may bind the S2 domain of the S-protein. Again, where there is S-protein or Coronavirus present in the original sample, this stage of the assay will yield a complex comprising the first binding agent (immobilised to a nanoparticle) and the second binding agent.

The lateral flow assay may further comprise a test line. The test line may comprise an immobilised agent which binds the capture moiety of the second binding agent. The immobilised agent may be described as a capture agent—where the capture agent is formulated to bind to the capture moiety of the second binding agent. Accordingly the test line (of the lateral flow assay) may comprise a capture agent.

The capture agent may comprise a moiety which binds to (or which has specificity and/or affinity for) the capture moiety of the second binding agent.

Where the capture moiety of the second binding agent comprises biotin (i.e. the second binding agent is biotinylated), the capture agent may comprise an agent which binds to biotin.

The capture agent may comprise polystreptavidin (PSA) or streptavidin.

The control may comprise a moiety which binds ACE2. This moiety may be immobilised to a control line. Any Ace2 which has not been bound by spike protein will bind to the binding moiety. If the Ace2 is bound to a detectable nanoparticle (for example a coloured nanoparticle) the control line will become visible as the detectable/coloured nanoparticles accumulate at the control line.

In view of the above, the disclosure provides a lateral flow assay for detecting Coronavirus or a Coronavirus antigen in a sample, said lateral flow assay comprising:
a first Coronavirus/Coronavirus antigen binding agent; and
a second Coronavirus/Coronavirus antigen binding agent;
wherein the first binding agent is not an antibody.

As stated, the lateral flow assay may further comprise a capture agent which comprises a moiety which binds to the second Coronavirus/Coronavirus antigen binding agent,

In one teaching, the disclosure provides a lateral flow assay for detecting Coronavirus or a Coronavirus antigen in a sample, said lateral flow assay comprising:
a nanoparticle conjugated ACE2; and
a biotin conjugated antibody which binds the S2 domain of the Coronavirus S-protein.

The lateral flow assay may further comprise a capture agent which comprises a moiety which binds biotin.

In use, a lateral flow assay of this disclosure may receive a sample, for example a sample of blood, saliva, nasal fluid, mucus, including nasal mucus, sweat, or faeces, or combinations thereof.

The sample may be brought into contact with the first binding agent—which binding agent (as described herein) binds to Coronavirus or a Coronavirus antigen. The first binding agent may comprise ACE2 which binds to Coronavirus S-protein.

As stated, the first binding agent may be conjugated to some form of detectable label—e.g. a gold nanoparticle.

Any Coronavirus or Coronavirus antigen present in the sample will bind to the first agent. This forms Coronavirus/first binding agent complexes or Coronavirus antigen/first binding agent complexes.

The absorbent pad part of the lateral flow assay will draw any complexes through the substrate of the lateral flow assay. The substrate may comprise nitrocellulose.

Any complexes being drawn through the sample are then contacted with the second binding agent of the lateral flow assay. Since the second binding agent comprises an agent which also binds the Coronavirus or Coronavirus antigen (perhaps at a different site from the first binding agent), the second binding agent will bind any complexes (which complexes comprise Coronavirus or Coronavirus antigen) as they move through the lateral flow assay.

This forms a further, or secondary complex comprising (i) first binding agent; (ii) Coronavirus or Coronavirus antigen and (iii) second binding agent.

These secondary complexes are further drawn through the lateral flow assay by the absorbent pad towards the test line.

As stated, the second binding agent may comprise a (capture) moiety which is bound by the capture agent present in the test line. Therefore as the secondary complexes are drawn through the sample and come into contact with the capture agent immobilised at the test line, the secondary complexes are also immobilised.

Immobilisation of the secondary complexes at the test line causes the appearance of a line—which line can be seen by virtue of the optically detectable label present on the first binding agent.

The presence or appearance of a line may mean that the sample has been obtained from a subject that has or has had a Coronavirus infection of a disease caused or contributed to by a Coronavirus.

Prior to use, a sample may be subject to some preparation or activation protocol.

The sample may, for example be treated by the addition of reagents and buffers. These buffers may neutralise certain enzymes and/or may increase or facilitate the flow of a sample into and through and assay.

A sample may be subject to a viral lysis protocol before use. For example, the sample may be treated with a composition designed to break up and/or lyse any coronavirus present in the sample. A protocol which breaks up or lyses the virus in a sample before use, may improve the sensitivity of the assay.

A sample to be tested may be contacted with an activating agent.

The activating agent may increase binding between any Coronavirus or Coronavirus antigen present in the sample and the first binding agent.

Where the sample is being tested for the presence of SARS-CoV-2, a SARS-CoV-2 S-protein, SARS-CoV or a SARS-CoV S-protein, the activating agent may increase binding between ACE2 (when used as the first binding agent) and the S-protein (or components thereof).

The activating agent may comprise a serine protease to prime the Coronavirus or any antigen thereof, for binding to the first agent.

The activating agent may comprise TMPRSS2 and/or trypsin.

The activating agent may comprise a serine protease inhibitor. The serine protease inhibitor may comprise, for example, TMPRSS2 or Furin. Inhibitors of this type may be added to the sample prior to use in a method or assay of this disclosure.

As such, this disclosure provides a method for treating a sample for use in a method of detecting a Coronavirus or Coronavirus antigen in the sample, said method comprising contacting the sample with a serine protease, trypsin or TMPRSS2. The protease (for example) may be used at a concentration of about 32-0.25 nm.

In one teaching, a sample may be contacted with a Mcllvaine buffer supplemented with 0.3% DDAO at a neutral pH (for example, pH 7.4).

Other buffers supplemented with surfactants such as, for example, tween 20 0.1-0.5%, tween 80 0.1-0.5%, EDTA 0.1-0.5% and poloxamers 0.1-0.5% in PBS or Mcllviane may also be used. Further, a method of detecting Coronavirus or a Coronavirus antigen in a sample may comprise

(i) providing a sample to be tested

(ii) optionally treating the sample with a protease, serine protease or TMPRSS2:

(iii) contacting the sample with a first Coronavirus/Coronavirus antigen binding agent under conditions which permit binding between the binding agent and any Coronavirus or Coronavirus antigen present in the sample; and

(iv) detecting binding agent/Coronavirus complexes and/or binding agent/Coronavirus antigen complexes;
wherein the first Coronavirus/Coronavirus antigen binding agent is not an antibody.

Any of the methods of this disclosure may be further used to diagnose or triage Coronavirus infections and/or disease, including for example COVID-19 in a point of case or point of need setting. The methods may be (as stated above), applied to samples obtained form or provided by symptomatic and/or asymptomatic subjects.

A sample may be further treated before being subjected to a method of this disclosure. For example, the pH of a sample may be adjusted. For example the pH of the sample may be adjusted so that it is above about pH8.

All of the methods of this disclosure may be combined or performed simultaneously or concurrently with other methods for the detection or diagnosis of other infections, antigens and/or diseases. The methods may be combined and/or performed simultaneously or concurrently with other immunoassays.

In other cases, the methods of this disclosure may find application in the research setting where they may be used to determine the binding affinity/kinetics/activity of a virus—for example a Coronavirus.

The methods of this disclosure may be used to determine the efficacy of particular drug candidates. For example prior to addition of the first binding agent (or concurrently therewith) a test agent may be added. That test agent may compete the first agent for binding to the Coronavirus and/or Coronavirus antigen. Where a test agent is found to compete with the first agent, that test agent may be useful in the treatment and/or prevention of Coronavirus infections and/or diseases or conditions associated with a Coronavirus infection. Potentially useful test agents may be identified as a negative result in an assay of this disclosure where the sample used is known to contain Coronavirus or a Coronavirus antigen.

### SEQUENCE LISTING

The instant application contains a Sequence Listing which has been submitted electronically in ASCII format and is hereby incorporated by reference in its entirety. Said ASCII copy, created on May 7, 2021, is named PG449449W0-766386-000001-NP-SequenceListing.txt and is 26 kilobytes in size.

### DRAWINGS

The patent or application file contains at least one drawing executed in color. Copies of this patent or patent application publication with color drawing(s) will be provided by the Office upon request and payment of the necessary fee. The present invention will now be described by reference to the following figures which show:

FIG. 1: A) positive line indicating the presence of biotinylated S1 antibody, B) Biotinylated Rabbit IgG was used for control.

FIG. 2: Visual representation of 100 uL of BSA blocked solutions of S1 polyclonal antibody or S1 antibody conjugated with GNP 40 nm OD=3.3 A) Unsuccessful conjugation as an indication of gold NP crashing out after addition of polyclonal S1 antibody B) Successful conjugation of monoclonal S1 antibody as gold remains read after addition of the antibody.

FIG. 3: Titration of S-RBD into 10 uL an 20 uL of ACE2-GNP OD=3.6 using halfrate 0.5 mg/mL PSA nitrocellulose membrane. S-RBD (0.25 mg/mL) was diluted 1:10000. 20-2.5 uL (test 1-5) of 1:10000 S-RBD was mixed with 2 uL of 1:50 dilution of (1 mg/mL) biotinylated S1 mab. 10 or 20 uL was added to the sample and the mixture was incubated for 15 min before running the strip. Sample was run through the strip and washed with 10 uL of drying buffer.

FIG. 4. Schematic diagram demonstrating the sensitivity of the lateral flow assay. A) demonstration of the sandwich formation after 2-15 min incubation of a solution prepared for wet assay followed by a subsequent run and PSA plotted assay strip, B) demonstrate the assay in absence S-RBD, C) indicates a successful asymmetric sandwich assay in presence of S-RBD protein, D) Test strip format containing 1 line of 0.5-2 mg PSA in 1% Sucrose, 1% PBS in filtered water.

FIG. 5: Schematic illustration of A) Wet assay of Viral protein, B) Lateral flow assay format demonstrating viral antigen-NP and S-RBD specific antibody sandwiched around the virus, C) Lateral flow assay with viral antigen-NP and antibody sandwiched around the viral protein.

FIG. 6: Comparison of A) Wet assay with B) dry assay.

FIG. 7: Comparison of asymmetric assay with S1 protein present versus S-RBD protein.

FIG. 8: A) Buffer RBD (50:50), 15 pg/ml OD20 gold colloid. 5 minutes incubation w/10 minute run, n=3, error=SD: B) Spiked saliva with RBD (50:50), OD20 gold 15 μg/ml using 8 μl deposition, 1% T20 PBS-B running buffer, 20 minute run time.

FIG. 9: Comparison of spiked saliva sample application: 15 μg/ml OD20 gold 4 μl dried.

FIG. 10: Comparison of gold volumes, 10 and 20 minute read times, RBD saliva spike 250 ng/ml (50:50)

FIG. 11: spiked saliva with RBD (1;1), OD20 gold 15 μg/ml using 8 μl deposition, 1% T20 PBS-B running buffer, 20 minute run time.

FIG. 12: cartoon showing the arrangement at the test line of an example lateral flow assay. In this figure, nitrocellulose strip 16 contains a test line having immobilised thereof a quantity PSA (14) immobilised thereon. In previous steps, a nanoparticle (NP) having immobilised thereof a quantity of ACE2 (2: or a Coronavirus/S-protein binding fragment thereof) is contacted with a sample thought to contain either Coronavirus and/or the Coronavirus S-protein under conditions which permit binding between any Coronavirus and/or the Coronavirus S-protein present in the sample and the ACE2 bound to the nanoparticle. In this figure, the antigen (the Coronavirus S-protein) is represented by components 4 (the RBD domain), 6 (the S1 domain) and 8 (the S2 domain). The result of this step of the assay (if the sample contains the target antigen (the S-protein)) is the formation of a complex between the S-protein (specifically the S1 domain thereof) and the ACE-2 component bound to the nanoparticle. The assay system further comprises an antibody (10) with specificity for the S2 domain of the S-protein (8). That antibody (10) is biotinylated (12). The assay permits the formation of a further complex with antibody 10. This complex is then captured at the test line by binding between the biotin moiety (12) of the antibody (10) and the PSA (14) at the test line (18). Because a test line contains multiple PSA (14) moieties, multiple nanoparticles become immobilised and can be detected. The method of detection may vary depending on the type of nanoparticle, but it may be optically detectable—the nanoparticle being a coloured bead or the like.

FIG. 13: Cartoon showing an exemplar assay format. In this case, the Coronavirus spike protein is shown as comprising three domains, the RBD domain (42), the S1 domain (44) and the S2-domain (46). In this assay, a sample (20) comprising spike protein is applied to a device which comprises a nitrocellulose strip (21). The strip (21) comprises a sample pad (22) for receiving the sample, a conjugation pad (24) comprising a first spike protein binding agent (26) and a second conjugation pad (28) comprising a molecule which itself comprises a nanoparticle (32) with ACE2 (30) (or a spike binding fragment thereof) immobilised thereto.

The sample (20) moves through the nitrocellulose strip (21) by capillary action/wicking. The sample maybe ‘pulled’ or drawn through by an absorption pad located at a distal point of the strip (21: not shown). The sample 20 and any spike protein therein (42, 44, 46) arrives at conjugation pad (24) which comprises an antibody (26) with specificity for the S2 domain (46) of the spike protein. In this example, the antibody is biotin labelled. The result of this interaction is the formation of a spike protein (42, 44, 46)::antibody complex (26). This complex will then move through to conjugation pad 2 (28) which comprises the Ace2 (30) bound nanoparticle (32). At conjugation pad 2 (28) there will form a further complex comprising not only the spike protein (42, 44, 46) and antibody (26), but also the Ace2 (30) bound nanoparticle (32). This larger complex will then continue to move through the strip (210 towards the text line (34) and control line (36). Test line (34) may have immobilised thereon s quantity of PSA—this will bind the biotin moiety of the antibody (26). The control line (36) may comprise an Ace2 binding moiety (38). In this regard, a test line forms because of complex binding to the PSA of the test line (via biotin on the anti-S2 antibody (26)) free ACE2/nanoparticle conjugates (30/32) bind to moiety (38) at the control line to form a red line.

### DETAILED DESCRIPTION

### Example 1

Biotinylation of antibodies specific to S-RBD is prepared by using Lightning-Link @ Rapid Biotin Conjugation Kit (Type B) following advised protocol:
1. Addition of 1 uL of LL Rapid Modifier reagent for each 10 uL of S1 antibody to be labeled and mixed.
2. Add the mixture of the mixture of S1 antibody and LL Rapid Modifier reagent was added to the lyophilised LL Rapid mix and redispersed with a pipette.
3. The sample was left for 15 min at room temperature (21 C).
4. After incubation 1 uL of Rapid Quencher reagent was added for every 10 uL of antibody used. The sample was left for 5 min and subsequently stored at 4 C until use.
5. The biotinylation was checked using a biotinylation test kit supplied by abcam.

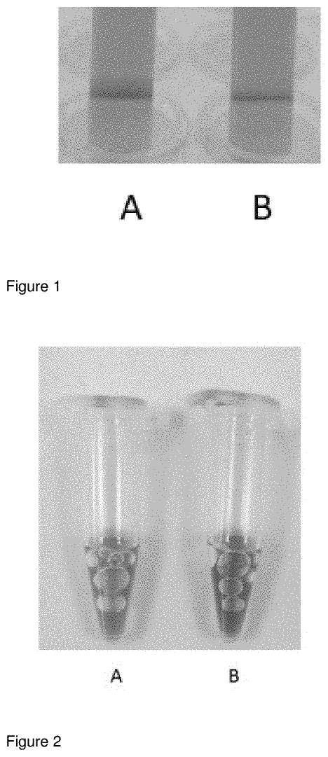

See also FIG. 1.

Gold Antibody Conjugate Buffer Optimisation

In initial studies the passive conjugation of NSP3, polyclonal S1 antibody, ACE-2, S1 antibody and CD147 on gold nanoparticles was optimised. This aims to select the conjugate with the highest specificity and sensitivity. These studies were carried out by mixing 1.5 uL—2.5 uL of antibody/antigen with conjugation buffers Tris, Borate, MES, TAPS and BES at pH ranges 5-10. Then 200 uL of gold nanoparticles were added to the mixtures. The samples were left on a shaker for 10 min at 21 C and analysed by eye and the optimal aggregation ratios were obtained by taking the ratio of 550 and 600 nm absorption intensity obtained by UV-vis. See FIG. 2.

TABLE 1
Gold aggregate ratios obtained for ACE-2 (1.5 uL added) and S1pAB (1.5 uL, 2.0 uL and 2.5 uL added).

| | Borate | Borate | Borate | | | | | | | | Borate | | |
|---|---|---|---|---|---|---|---|---|---|---|---|---|---|
| | 9.3 | 9.0 | 8.5 | MES6.7 | MES5.3 | TES7.1 | BES7.8 | TES7.5 | TAPS8.5 | BES6.5 | 9.24 | TES7.1 | |
| ACE-2 | 3.8630849 | 3.974217 | 4.01119 | 4.05075 | 4.1286 | 3.95833 | 3.91091 | 3.90235 | 4.0814 | 4.08818 | 4.2164329 | 4.04699 | 1.5 uL |
| S1pAB | 3.2467018 | 2.777533 | 2.92532 | 2.71992 | 2.27637 | 2.45236 | 2.8516 | 2.74611 | 3.03073 | 2.55753 | 3.1649485 | 2.88229 | 1.5 uL |
| | 3.5624123 | 3.250627 | 3.30869 | 2.71919 | 1.57018 | 2.4276 | 3.00229 | 2.68333 | 3.34241 | 2.20983 | 3.4888889 | 2.71013 | 2.0 uL |
| | 3.4193989 | 3.45 | 3.1788 | 2.81142 | 2.02534 | 2.34347 | 3.07317 | 2.64666 | 3.18017 | 2.16944 | 3.5169492 | 2.74587 | 2.5 uL |
| Gold | 4.4733475 | 4.433476 | 4.35759 | 4.46316 | 4.40421 | 4.21937 | 4.27912 | 4.23633 | 4.31174 | 4.30498 | 4.2409639 | 4.22088 | 0 uL |
| NP | | | | | | | | | | | | | |

The numbers in bold font represent successful aggregation with a 550 nm/600 nm UV vis ratio [symbol] above 3.5, the numbers in italic font represent 550/600 nm ratios 3-3.49 and the numbers in underlined font are unsuccessful conjugation. MES, TES, TASPS and Borate represents the conjugation buffers added as 5 uL and the gold NP used is 40 nm with OD = 5.

TABLE 2
Gold aggregate ratios obtained for NSP3 (0-2.0 uL added) and CD147 (0 uL-2.0 uL added).

| | Borate | Borate | Borate | | | | | | | | | |
|---|---|---|---|---|---|---|---|---|---|---|---|---|
| | 9.3 | 9.0 | 8.5 | MES6.7 | MES5.5 | TES7.1 | BES7.8 | TES7.5 | TAPS8.5 | BES6.5 | TES7.1 | |
| NSP3 | 4.342975 | 4.38758 | 4.378723 | 4.446623 | 4.398693 | 4.390558 | 4.277551 | 4.324324 | 4.305263 | 4.26506 | 4.288066 | 0 uL |
| | 4.208835 | 4.167323 | 4.201597 | 4.13852 | 4.136719 | 4.223735 | 4.050373 | 4.099065 | 4.15458 | 4.125 | 3.970534 | 0.5 uL |
| | 4.286885 | 4.009328 | 4.129482 | 4.083969 | 4.128352 | 4.285132 | 4.322034 | 3.979817 | 4.256674 | 4.07457 | 4.27789 | 1.5 uL |
| | 3.99812 | 4.033962 | 4.046422 | 4.092453 | 4.100569 | 4.293634 | 4.330526 | 4.313402 | 4.280665 | 3.59271 | 4.403846 | 2.0 uL |
| CD147 | 3.88764 | 4.322034 | 4.324324 | 4.409766 | 4.41649 | 4.374732 | 4.261224 | 3.227205 | 3.360601 | 3.851779 | 3.265139 | 0 uL |
| | 1.895075 | 2.602378 | 2.087659 | 1.893548 | 1.88609 | 2.191638 | 1.7892 | 2.04662 | 1.913734 | 1.703264 | 1.689616 | 0.5 uL |
| | 4.4375 | 4.118 | 4.442731 | 4.345041 | 4.481319 | 4.129845 | 4.383475 | 4.331959 | 4.420601 | 4.352321 | 4.33543 | 1.5 uL |
| | 1.060606 | 1.057143 | 1.057143 | 1.057143 | 1.057143 | 1.057143 | 1.055556 | 1.051282 | 1.046512 | 1.083333 | 1.088235 | 2.0 uL |

The numbers in bold font represent successful aggregation with a 550 nm/600 nm UV vis ration above 3.5, the numbers in italic font represent 550/600 nm ratios 3-3.49 and the underlined numbers are unsuccessful conjugation. MES, TES, TASPS and Borate represents the conjugation buffers added as 5 uL and the gold NP used is 40 nm with OD = 5.

TABLE 3
Gold aggregate ratios obtained for Spike S1 AB (0-2.5 uL added), NSP3 antibody (2.5 uL added) and CD147 (2.5 uL added).

| | Borate | Borate | Borate | | | | | | | | | |
|---|---|---|---|---|---|---|---|---|---|---|---|---|
| | 9.3 | 9.0 | 8.5 | MES6.7 | MES5.5 | TES7.1 | BES7.8 | TES7.5 | TAPS8.5 | BES6.5 | BES6.5 | |
| Spike S1 AB | 4.462687 | 4.444444 | 4.32766 | 4.4267 | 4.39957 | 4.44541 | 4.49666 | 4.47682 | 4.42217 | 4.46154 | 4.42117 | 0 uL |
| | 3.858736 | 3.414089 | 3.570191 | 2.01941 | 3.40193 | 3.39187 | 3.43954 | 3.20821 | 3.58944 | 3.85316 | 3.08309 | 0.5 uL |
| | 4.298174 | 4.223969 | 4.246377 | 4.30769 | 4.32985 | 4.34232 | 4.39195 | 4.28016 | 4.34728 | 4.13917 | 4.25355 | 1.5 uL |
| | 4.353814 | 4.346639 | 4.347639 | 4.42949 | 4.41365 | 4.3264 | 4.43226 | 4.38205 | 4.23434 | 4.27216 | 4.14147 | 2.0 uL |
| NSP3 | 4.473913 | 4.412527 | 4.399142 | 4.4586 | 4.39019 | 4.41432 | 4.31818 | 4.21138 | 4.202 | 4.23926 | 4.27071 | 2.5 uL |
| CD147 | 1.918098 | 1.66581 | 1.541568 | 1.66884 | 1.62973 | 1.58495 | 1.63464 | 1.60638 | 1.74668 | 1.53791 | 1.58945 | 2.5 uL |
| Spike S1 AB | 1.561381 | 3.842004 | 1.519303 | 1.70496 | 1.66279 | 1.75232 | N/A | N/A | N/A | N/A | N/A | 2.5 uL |

The numbers in bold font represent successful aggregation with a 550 nm/600 nm UV vis ration above 3.5, the numbers in italic font represent 550/600 nm ratios 3-3.49 and the underline numbers are unsuccessful conjugation. MES, TES, TASPS and Borate represents the conjugation buffers added as 5 uL and the gold NP used is 40 nm with OD = 5.

Protocol for Passive Conjugation of Antigens and Antibodies to Nanoparticles (Such as Gold Nanoparticles).

Passive conjugation of antigens and antibodies such as Angiotensin-Converting Enzyme 2 (ACE2), any versions of S-RBD and S1 antibodies in the volume range 0.5-5 uL to conjugation buffers (Tris, Borate, MES, TAPS and BES) in the pH ranges 2-10 where used in the quantity of 5 uL. After mixing the protein with the conjugation buffer we add OD1-OD10 gold nanoparticles in size ranges 10-150 nm (100-500 uL). The mixture was left on a shaker for 45 min at room temperature (21 C). 1 uL of (300 mg/mL) of Probumin was added to the mixture and vortexed. Then left for 30 min at room temperature (21 C). Then 900 uL drying buffer (pH range >6, 1 mM Borate, 2-5% sucrose, 1-3% BSA, 0.1-2% Tween 20 or Tween 80, 0.1% sodium azide in filtered deionised water) was added to make up 1 mL in an eppendorf tube and the sample was centrifuged for 10-15 min at 4000 g. Then 950 uL of the supernatant was removed and 950 uL of drying buffer was added. The sample was centrifuged for 10-15 min at 4000 g. Then 950 uL of sample was removed and 50 uL of drying buffer was added. The sample was vortexed and stored at 4 C until use.

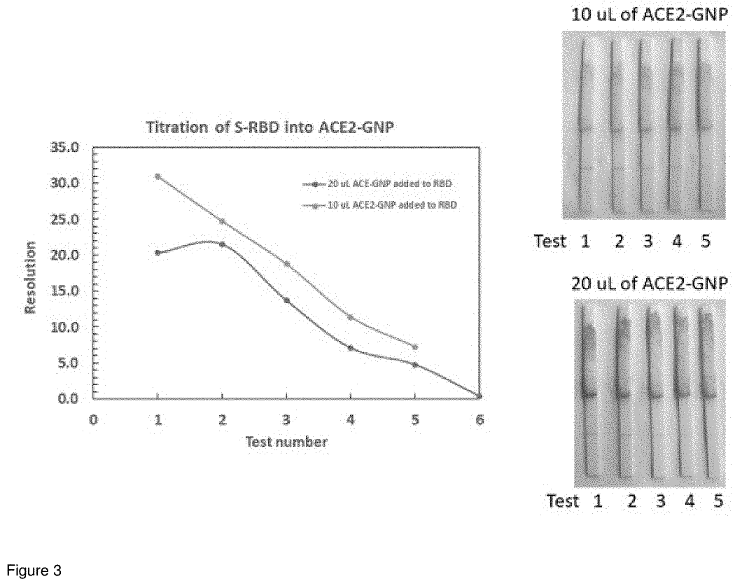

Plotting of PSA on nitrocellulose strips was performed by preparing a solution of 4.7 uL of 4.36 mg/ml of PSA, 4.1 uL of 10% sucrose, 3.6 uL of PBS buffer and 29 uL filtered and deionised water. The mixture was used to plot a line on 30 cm nitrocellulose membrane CN140 with a plotting rate of 0.1 mm/s. The membrane was dried at 40 C and attached to a card together with an absorbent pad. The membrane was cut in 3 mm to yield 100 strips. See FIG. 3.

### Example 2—Parts of a Useful Assay May Include

1. Passive conjugation of SARS-CoV2 specific antigens such as, ACE-2, to nanoparticles such as gold nanoparticles.

2. Biotinylation of S-RBD antibodies.

3. Plotting of 0.5-2 mg/mL of PSA, S-RBD antibodies or antigens, biotinylated S-RBD antibodies or antigens to nitrocellulose strips.

4. Spraying a solution of ACE-2 or SARS-CoV2 specific antigens with or without TMPRSS2 protein or other serine proteases.

5. Procedure for the formation of asymmetric sandwich. Mix together 20-2.5 uL of saliva, serum or throat swab containing S-RBD, S1+S2 and S1 protein solution in 0-20 uL drying buffer with 2 uL of conjugated S-RBD specific antibodies and 5-20 uL of ACE-2 conjugated to gold nanoparticles 40 nm.

6. Add to the nitrocellulose strips and if the saliva, serum or throat swab contained viral proteins then a line appears on the strip.

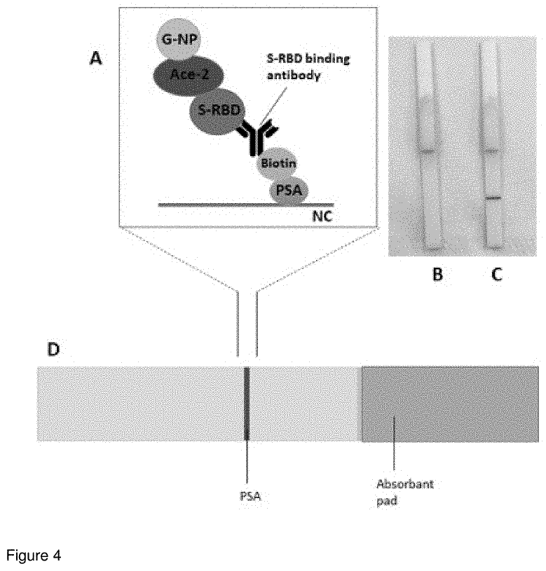

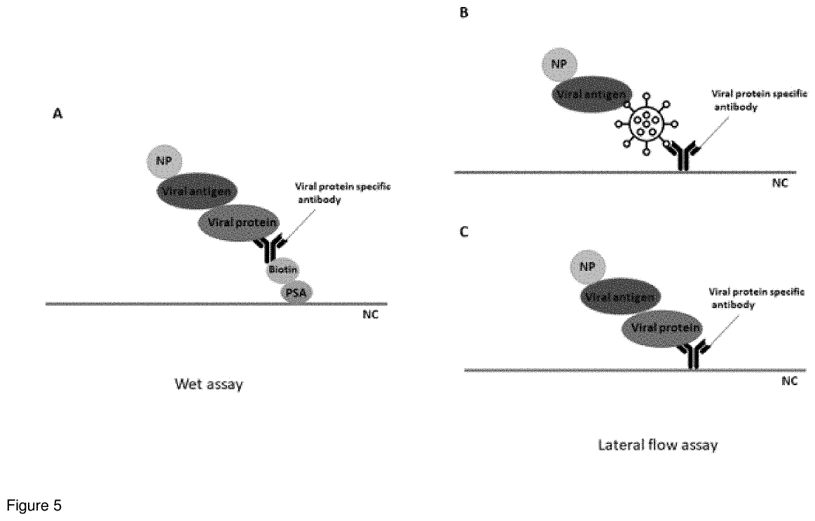

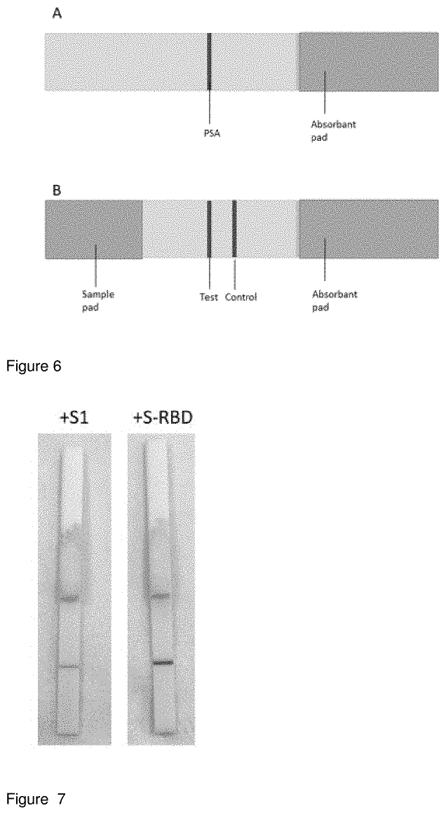

See FIGS. 4, 5 and 6 for additional details.

Sample/Antigen Activation with TMPRSS2

Experiments show that the open (activated) form (S-RBD) binds stronger than S1 to ACE2 in lateral flow assay. See FIG. 7. Without wishing to be bound by theory, when TMPRSS2 is present, it cleaves a residue on S1 that leads to a conformational change that results in the S-RBD binding site to get exposed and bind tight to ACE2. TMPSSR2 could be replaced by a whole range of different proteases that will give the same results.

Optional Features
1. The amount and type of gold nanoparticle could be modified such that the intensity of the band generated in the presence of SARS-COV-2 proteins increased more progressively based on the viral protein concentration. This would allow the LFA to be used to measure how well another compound binds to ACE2 in competition with viral proteins. This could be used to test potential drug candidates for treatment of SARS-COV-2 rapidly and cheaply compared to other lab based techniques (mostly ELISA). Note current formulation designed to give as much signal as possible with as little viral protein as possible.
2. This LFA setup described is likely to be applicable to other viruses (other than SARS-COV-2). As viruses all need to bind to some protein to gain entry into cells such a protein might substitute for ACE2 and along with an antibody specific to alternative virus the same type of sandwich LFA could be constructed.

The methods as described herein could be further modified as follows:

1. Both the second binding agent (for example the specific antibody) as well as first binding agent (e.g. angiotensin-Converting Enzyme 2 (ACE2)) could be replaced with other types/versions of antibodies. All that would be required is that there were two separate binding sites where the new/alternative antibodies could bind. Specific binding domains (e.g. short peptides/aptamers/etc.) could be used instead of whole antibodies/proteins. This could improve overall device stability and potentially binding affinity.

2. Gold nanoparticles (an option for tagging or labelling the first binding agent) could be changed to a wide range of other materials. Other metals could potentially be used. Also, other fluorescent or coloured compounds are potential substitutes. Any nanoparticles could be used.

3. The test could be arranged in a variety of formats and conjugations. For example, the methods of this invention could be adapted to take place in ELISA format, ELIspot format, as Dot blots, radioimmunoassays and the like. Additional information regarding immunoassays which may be adapted or used to permit the detection of Coronavirus and/or Coronavirus antigens in samples may be found in The Handbook of Immunoassay technologies, (Vashist and Luong (2018), published by Academic press: see in particular chapter 1: Immunoassays: an overview: the contents of which is incorporated herein by reference.

### Example 3

### Summary

The titration of RBD in an assay with a gold conjugated ACE2 component as a signalling moiety and a human monoclonal anti-SARS-CoV-2 Spike antibody as the capture moiety on a nitrocellulose format has been completed.

The wet assay format has been optimised into a dry assay format. Moreover, sensitivity has been optimised by titrating RBD, attenuated SARS-CoV-2 or SARS-CoV-2 viruses spiked into negative saliva samples on the dry assay format to evaluate the assay performance.

The assay is evaluated using clinical samples to confirm those results obtained from the abovementioned mentioned titrations.

Assay Components:
1 mg/ml of human monoclonal anti SARS-CoV-2 Spike antibody.
CN140 nitrocellulose membrane prepared as a 60 mm height and 5 mm broad strip
Sink pad
Conjugation pad (GF conjugate pad)
Backing card
Sample pad
40 nm OD20 gold colloid conjugated with 15 μg/ml ACE2 with 20 mM MES pH 5.3)

Plotting Procedure:

1 mg/ml of human monoclonal anti SARS-CoV-2 Spike antibody is plotted on a CN140 membrane with a rate of 0.1 uL/mL to form a test line.

Spray Procedure:

40 nm OD20 gold colloid conjugated with 15 μg/ml ACE2 with 20 mM MES pH 5.3 was prepared and sprayed onto the conjugation pad.

Strips were closed using 1-well Kanani housing tops and bottoms and passed through a cassette sealer.

The devices were placed in a labelled aluminium pouch with sufficient desiccant before sealing.

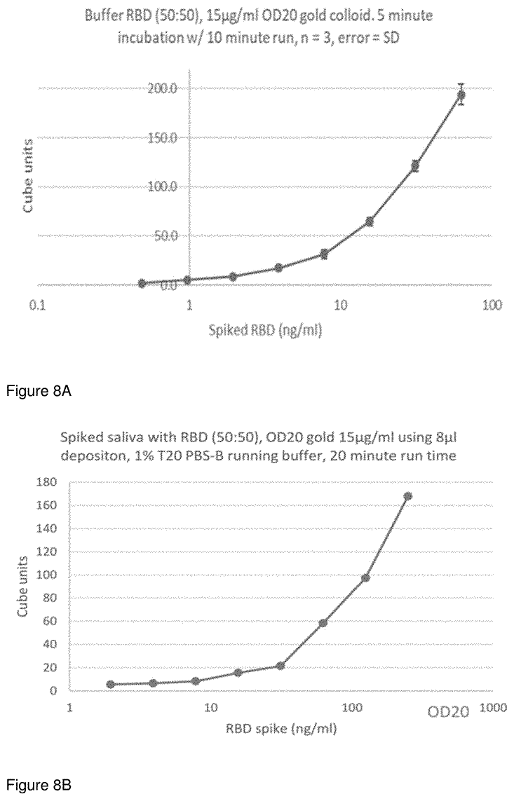

Procedure for Run of Dry 40 nm ACE-2 Gold Assay Using Healthy/Negative Saliva Spiked with RBD:
The concentration of gold is at OD20 15 μg/ml ACE2 and 8 μl of the material was deposited centrally onto the conjugate pad.
RBD spikes are diluted to the required concentration, or if testing with saliva, the initial concentration is doubled as RBD and saliva are combined 50:50.
Sample/saliva is deposited onto the dried conjugate using 20 μl and an incubation time of 5 minutes and 20 min (see FIGS. 8A and 8B).

Procedure for Run of Dry ACE2 Gold Assay Spiked with Saliva Spiked with Attenuated Virus:
Strips ran with 80 μl of 1×PBS, 1% Tween 20, 1% BSA. (other buffers have also been used in the optimisation process)
Gold is at OD20 (15 μg/ml ACE2 from R&D); 8 μl deposited centrally onto the conjugate pad.
Heat killed and irradiated virus stored at −80° C.
Diluting irradiated virus requires 5×PBS 1% BSA 1% Pluronic 68. This buffer was also used as the running buffer.

Optimisation of the Sample and Gold Colloid Volume (for Saliva)
Saliva was collected and filtered through a 0.45 μm filter
Spiked with RBD to final concentration of 250 ng/ml (50:50)
15 μg/ml OD20 gold colloid dried (4-10 μl) on conjugate pad
Compared 20, 40, 60, 80 μl sample volume, 5 minute incubations
Assay ran with 80 μl running buffer

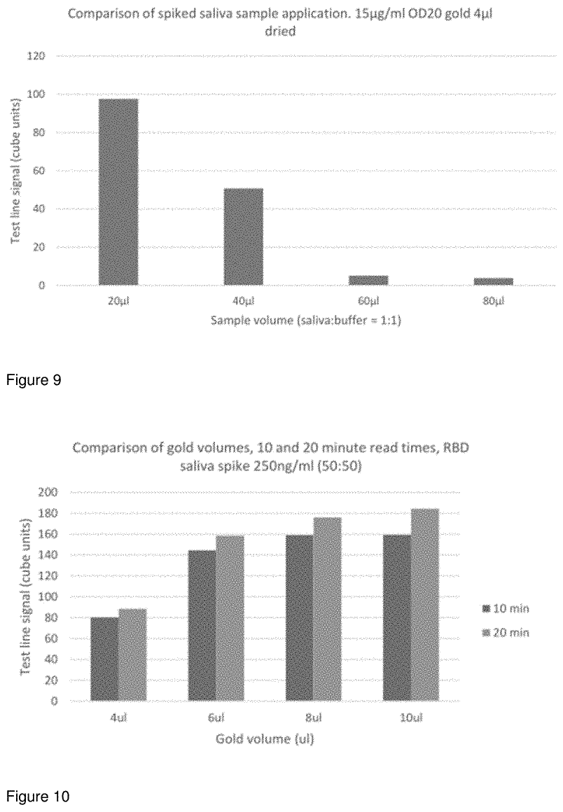

Assay Procedure:
20 μl is the current sample volume used for assay testing.
Increasing the sample volume allows the assay to run, however this does not increase the signal.
Larger conjugate pad possibly required to allow suitable incubation time. See FIG. 9.

Evaluation of Gold Volume Effects (Saliva)
Repeated previous assay format for spiked saliva
Dried varying volumes of gold: 4, 6, 8, 10 μl
20 μl spiked saliva with a 5-minute incubation, ran with 80 μl running buffer comparing
10 and 20 minute read times

The conclusion from this work is that 8 ul gold gives the optimal signal; 6 ul gold is also useful as the signal it generates is not too far below. See FIG. 10.

Assessment of Assay Sensitivity with Saliva
Dried 8 μl of 15 μg/ml OD20 colloid
Prepared saliva spiked with a starting concentration of 250 ng/ml RBD; doubling
dilutions down.
5 minute incubation with 20 μl sample (saliva:buffer; 1:1), 80 μl running buffer run and
read after 20 minutes.

| ng/ml | Cube units |
|---|---|
| 250 | 168 |
| 125 | 97.4 |
| 62.5 | 58.2 |
| 31.3 | 21.5 |
| 15.6 | 15.5 |
| 7.8 | 8.3 |
| 3.9 | 6.6 |
| 2 | 5.5 |

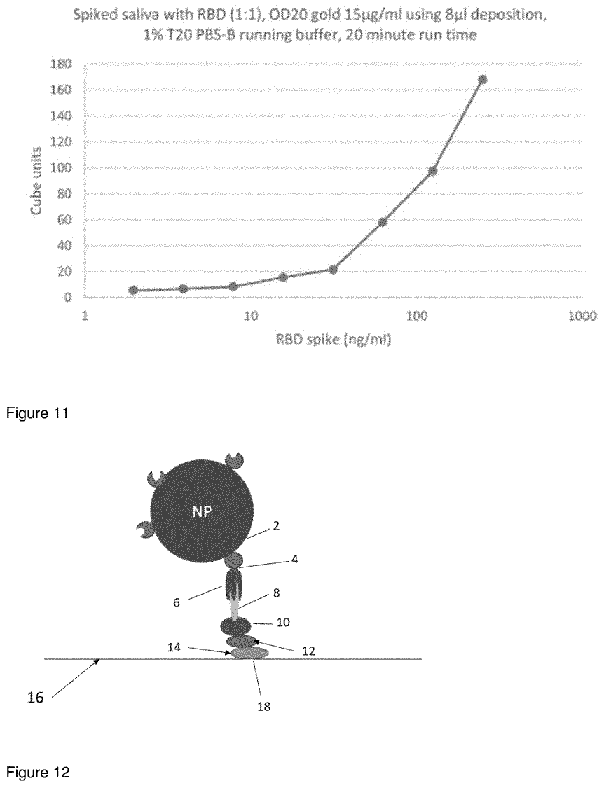

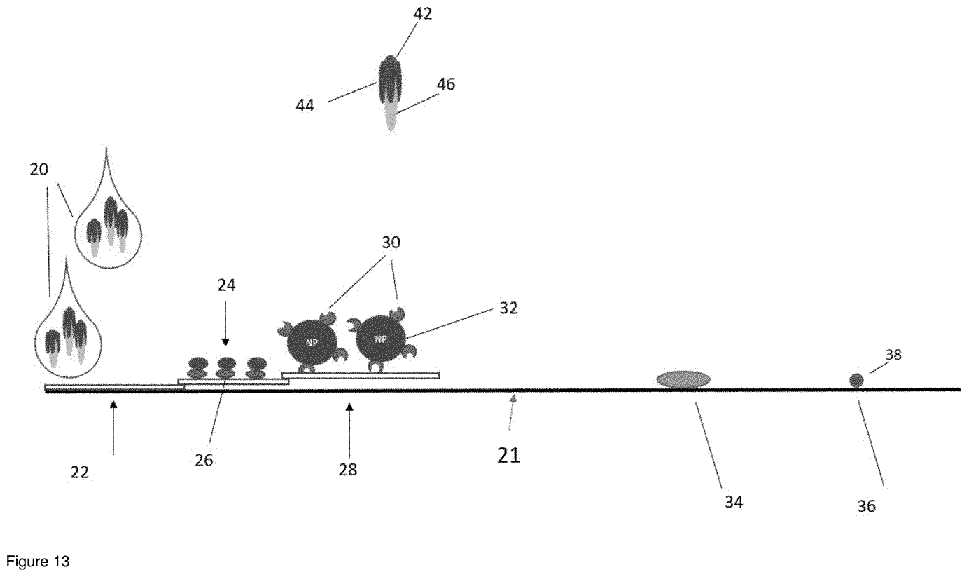

See also, FIG. 11.

### Example 3: Spiking of Fresh Virus into Saliva

Materials

SARS-CoV2 virus, drooled saliva, universal buffer, 12 SARS-CoV-2 Ag tests (as disclosed herein), eppendorf tubes, pipettes, tips and a timer.

Experimental Procedure

After addition of virus (used at 3×10⁴ PFU/mL) to the saliva, the sample was vortexed and inverted to ensure appropriate mixture to create a homogeneous sample. Dilutions were performed as follows to make up 5×10³, 2×10³ and 5×10² and 0 pfu/mL from the provided 3×10⁴ PFU/mL viral stock solution as follows.
400 uL of universal buffer was added to 200 uL of the viral spiked samples and the sample was incubated for 60 s.
120 uL of the samples were added to a sample well and the device was left to run for 15 min. The device was read in the reading frame of 15-30 min and results were read by eye.

Titration of 3×10⁴ PFU/mL Virus

Dilutions were performed as follows to make up 5×10³, 2×10³ and 5×10² and 0 PFU/mL from provided 3×10⁴ PFU/mL viral stock solution as follows:

Titration was completed as follows (Table 4):

TABLE 4

| | PFU/mL | Sample/uL | Saliva | Cube units |
|---|---|---|---|---|
| 1 | 0 | 0 | 200 uL | 2.2 |
| 2 | 5 × 10³ | 83.33 uL (from 3 × | 416.7 uL | 13 |
| | | 10⁴ PFU/mL) | | |
| 3 | 2 × 10³ | 33.33 (from 3 × | 466.7 uL | 11 |
| | | 10⁴ PFU/mL) | | |
| 4 | 5 × 10² | 8.33 uL (from 3 × | 491.7 uL | 3 |
| | | 10⁴ PFU/mL) | | |
| 5 | 2 × 10² | 3.33 uL (from 3 × | 496.7 uL | 1.7 |
| | | 10⁴) | | |

Titration was performed in singles and a limit of detection of 5×10² PFU/mL was visible.

Second titration on 20 healthy saliva samples were performed.

Cross Reactivity Studies

Materials

SARS-CoV-2 isolate, Culture materials, General consumables: filter tips, micropipette, vortex, PPE, reagent reservoirs, towels etc., Category 3 facilities and PPE, Human Coronavirus 229E, OC43, NL63, Influenza A and B, Rhinovirus and Adenovirus, 4 SARS-CoV-2 Rapid Antigen Tests devices (as described herein).

Protocol

This validation protocol will evaluate the specificity of the test by testing potentially cross-reactive microorganisms. The following microorganisms were tested in triplicate. The results were interpreted by two operators, each blinded to the result of the other. If a discrepant result was obtained, a third operator was called for a result. Invalid results are to be repeated once.

TABLE 5
The results of the Cross Reactivity assay

| | Conc. Of | | |
|---|---|---|---|
| Organisms likely in | microorganisms | | |
| circulating area | PFU/ml | Repeats | Results |
| Adenovirus | 1.00 × 10⁵ | 3 | Negative |
| Influenza A | 1.00 × 10⁵ | 3 | Negative |
| Influenza B | 1.00 × 10⁵ | 3 | Negative |
| hCoV229 | 1.00 × 10⁵ | 3 | Negative |
| hCovOC43 | 1.00 × 10⁵ | 3 | Negative |
| hCoVNL63 | 1.00 × 10⁵ | 3 | Negative |
| Rhinovirus | 1.00 × 10⁵ | 3 | Negative |

Antigen Tests devices (as disclosed herein), trimeric spike protein 1 mg/mL, Mcllvaine buffer with 0.3% DDAO pH 7.4.

1 mg/mL of Trimeric spike was diluted in saliva to make a 5 ng/mL solution.

100 uL of Mcllvaine buffer 0.3% DDAO pH 7.4 was added to 50 uL of the viral spiked samples and the sample was incubated for 60 s.

120 uL of the samples were added to a sample well and the device was left to run for 15 min.

The device was read in the reading frame of 15-30 min and results were read by eye.

1. A method for detecting Coronavirus or a Coronavirus antigen in a sample, said method comprising:
contacting the sample with a Coronavirus/Coronavirus antigen binding agent under conditions which permit binding between the binding agent and any Coronavirus or Coronavirus antigen present in the sample and detecting complexes comprising binding agent/Coronavirus/Coronavirus antigen, wherein the Coronavirus/Coronavirus antigen binding agent is not an antibody and detection of complexes comprising binding agent and Coronavirus/Coronavirus antigen, indicates that the sample contains Coronavirus and/or Coronavirus antigen and/or that the sample may have been provided by or obtained from a subject that has or has been infected with Coronavirus.

2. The method of claim 1, wherein the Coronavirus is SARS-CoV-2, SARS-CoV and/or MERS-CoV.

3. The method of claim 1, wherein the Coronavirus antigen is the spike (S)-protein, the S1 domain of the S-protein, the S2 domain of the S-protein and/or receptor binding domain (RBD) of the S-protein.

4. The method of claim 1, wherein the binding agent comprises ACE2 or a Coronavirus/Coronavirus antigen binding fragment thereof.

5. The method of claim 1, wherein the binding agent comprises a protein having a sequence of SEQ ID NO: 1, SEQ ID NO: 2 or a Coronavirus/Coronavirus antigen binding fragment thereof.

6. The method of claim 1 wherein the binding agent comprises DPP4 or a Coronavirus/Coronavirus antigen binding fragment thereof.

7. The method of claim 1, wherein the binding agent comprises a protein having a sequence of SEQ ID NO 3, SEQ ID NO: 4 or a Coronavirus/Coronavirus antigen binding fragment thereof.

8. The method of claim 1, wherein the binding agent is conjugated or bound to a nanoparticle.

9. The method of claim 8, wherein the nanoparticle is coloured.

10. The method of claim 1, wherein the sample comprises a biological fluid, saliva, blood and/or a fraction thereof.

11. The method of claim 1, wherein the sample is combined with a buffer prior to contacting the sample with a Coronavirus/Coronavirus antigen binding agent.

12. The method of claim 11, wherein the buffer comprises a detergent to disrupt any virus present in the sample.

13. The method of claim 1, wherein the method further comprises the use of a second Coronavirus/Coronavirus antigen binding agent.

14. The method of claim 13, wherein any step of adding second Coronavirus/Coronavirus antigen binding agent yields complexes which comprise the second binding agent and/or any Coronavirus/Coronavirus antigen.

15. The method of claim 14, wherein the second Coronavirus/Coronavirus antigen-binding agent is contacted with the sample.

16. The method of claim 15, wherein any step comprising the second Coronavirus/Coronavirus antigen binding agent is executed before, concurrently with or after any step comprising a or the first, Coronavirus/Coronavirus antigen binding agent.

17. The method of claim 16, wherein the step of adding a second Coronavirus/Coronavirus antigen binding agent is executed before the step of adding the a or the first Coronavirus/Coronavirus binding agent.

18. The method claim 13, wherein before the detecting step, the method comprises contacting the sample and/or any binding agent/Coronavirus complexes and/or first binding agent/Coronavirus antigen complexes, with the second Coronavirus/Coronavirus antigen binding agent.

19. The method of claim 13, wherein the second binding agent comprises a molecule selected from the group consisting of:
(i) ACE2 or a Coronavirus/Coronavirus antigen binding fragment thereof;
(ii) DPP4 or a Coronavirus/Coronavirus antigen binding fragment thereof;
(iii) SEQ ID NO: 1, SEQ ID NO: 2; SEQ ID NO: 3; SEQ ID NO: 4 or a or a Coronavirus/Coronavirus antigen binding fragment thereof;
(iv) an antibody which binds the spike (S)-protein, the S1 domain of the S-protein, the S2 domain of the S-protein and/or the receptor binding domain (RBD) of the S-protein.
(v) an antibody which binds the same target as the first binding agent; and
(vi) an antibody which binds the S2 domain of the S-protein

20. The method of claim 13, wherein the second binding agent is biotinylated.

21. The method of claim 13, wherein the second binding agent comprises an antibody which binds the S2 domain of the Coronavirus S-protein.

22. A method of for detecting Coronavirus or a Coronavirus antigen in a sample, said method comprising:
contacting the sample with a first Coronavirus/Coronavirus antigen binding agent under conditions which permit binding between the first binding agent and any Coronavirus or Coronavirus antigen present in the sample to form a first binding agent/Coronavirus or a first binding agent/Coronavirus antigen, complex, wherein the first Coronavirus/Coronavirus antigen binding agent is not an antibody;
contacting the sample and/or any complex, with a second Coronavirus/Coronavirus antigen binding agent under conditions which permit binding between any complexes and the second binding agent; and
detecting first binding agent/Coronavirus/second agent complexes and/or first binding agent/Coronavirus antigen/second agent complexes,
wherein detection of a first binding agent/Coronavirus/second agent complex and/or first binding agent/Coronavirus antigen/second agent complex indicates that the sample contains Coronavirus and/or Coronavirus antigen and/or that the sample may have been provided by or obtained from a subject that has or has been infected with Coronavirus or has had, a Coronavirus infection and/or a disease or condition associated therewith.

23. A method for detecting SARS-CoV-2 or a SARS-CoV-2 antigen in a sample, said method comprising:
contacting the sample with ACE2 or a SARS-CoV-2/SARS-CoV-2 antigen binding fragment thereof, under conditions which permit binding between the ACE2/ACE2 fragment and any SARS-CoV-2 or SARS-CoV-2 antigen present in the sample and detecting ACE2/ACE2 fragment::SARS-CoV-2 complexes and/or ACE2/ACE2 fragment::SARS-CoV-2 antigen complexes.

24. The method of claim 23, wherein the method further comprises the use of an antibody binding the S2 domain of the S-protein.

25. The method of claim 23, wherein the detection of ACE2/ACE2 fragment::SARS-CoV-2 complexes and/or ACE2/ACE2 fragment::SARS-CoV-2 antigen complexes, indicates that the sample may have been provided by or obtained from a subject that has or has been infected with SARS-CoV-2.

26. A method for detecting MERS-CoV or a MERS-CoV antigen in a sample, said method comprising:
contacting the sample with DPP4 or a MERS-CoV/MERS-CoV antigen binding fragment thereof, under conditions which permit binding between the DPP4/DPP4 fragment and any MERS-CoV or MERS-CoV antigen present in the sample and detecting DPP4/DPP4 fragment::MERS-CoV complexes and/or DPP4/DPP4 fragment::MERS-CoV antigen complexes.

27. The method of claim 26, wherein the detection of DPP4/DPP4 fragment::MERS-CoV complexes and/or DPP4/DPP4 fragment::MERS-CoV antigen complexes, indicates that the sample may have been provided by or obtained from a subject that has or has been infected with MERS-CoV.

28. A lateral flow assay for the detection of Coronavirus, a Coronavirus antigen, SARS-CoV-2, SARS-CoV, MERS-CoV, the S-protein of SARS-CoV-2, the S-protein of SARS-CoV or the S-protein of MERS-CoV in a sample, the lateral flow assay comprising:
a first Coronavirus binding agent, which is not an antibody; and
a second Coronavirus binding agent.

29. The lateral flow assay of claim 28, wherein the first binding agent comprises ACE2 or a Coronavirus, a Coronavirus antigen, SARS-CoV-2, SARS-CoV, MERS-CoV, SARS-CoV-2 S-protein, S-protein of SARS-CoV S-protein or MERS-CoV S-protein, binding fragment thereof.

30. The lateral flow assay of claim 28, wherein the first binding agent is conjugated to a nanoparticle.

31. The lateral flow assay of claim 28, wherein the nanoparticle is a coloured nanoparticle.

32. The lateral flow assay claim 28, wherein the second binding agent comprises an antibody which binds to a different domain of the Coronavirus spike protein to the first binding agent.

33. The lateral flow assay of claim 28, wherein the second binding agent comprises an antibody which binds the S2 domain of the Coronavirus spike protein.

34. The lateral flow assay of claim 28, wherein the lateral flow assay comprises an immobilised capture agent.

35. The lateral flow assay of claim 28, wherein the immobilised capture agent is immobilised at a test line of the lateral flow assay and comprises polystreptavidin (PSA).

36. The lateral flow assay of claim 28, wherein the second binding agent is biotinylated.

37. A lateral flow assay for the detection of Coronavirus or the S-protein of SARS-CoV-2, in a sample, the lateral flow assay comprising:
a Coronavirus binding agent comprising a nanoparticle with ACE2, or an S-protein or S1 domain binding fragment thereof, bound thereto; and
a Coronavirus binding agent comprising a biotinylated antibody which binds the S2 domain of the Coronavirus S-protein.

38. The lateral flow assay of claim 37, wherein the assay further comprises a test line comprising PSA.

39. The lateral flow assay of claim 37, wherein one Coronavirus binding agent is spaced apart from the other Coronavirus binding agent of the assay.

40. The lateral flow assay of claim 37, wherein the assay is configured to receive a sample and contact that sample with a biotinylated antibody which binds the S2 domain of the Coronavirus S-protein to yield antibody/Coronavirus or S-protein complexes.

41. The lateral flow assay of claim 40, wherein any antibody/Coronavirus or S-protein complexes assay are contacted with the nanoparticle to yield complexes comprising the antibody, nanoparticle and any Coronavirus or Coronavirus or S-protein.

42. A method of for detecting Coronavirus or a Coronavirus antigen in a sample, said method comprising:
contacting the sample with a first Coronavirus/Coronavirus antigen binding agent under conditions which permit binding between the first binding agent and any Coronavirus or Coronavirus antigen present in the sample to form a first binding agent/Coronavirus or a first binding agent/Coronavirus antigen, complex;
contacting the sample and/or any complex, with a second Coronavirus/Coronavirus antigen binding agent under conditions which permit binding between any complexes and the second binding agent, wherein the second Coronavirus/Coronavirus antigen binding agent is not an antibody; and
detecting first binding agent/Coronavirus/second agent complexes and/or first binding agent/Coronavirus antigen/second agent complexes, wherein detection of a first binding agent/Coronavirus/second agent complex and/or first binding agent/Coronavirus antigen/second agent complex indicates that the sample contains Coronavirus and/or Coronavirus antigen and/or that the sample may have been provided by or obtained from a subject that has or has been infected with Coronavirus or has had, a Coronavirus infection and/or a disease or condition associated therewith.

43. The method of claim 42, wherein the first binding agent is selected from the group consisting of:
(i) ACE2 or a Coronavirus/Coronavirus antigen binding fragment thereof;
(ii) DPP4 or a Coronavirus/Coronavirus antigen binding fragment thereof;
(iii) SEQ ID NO: 1, SEQ ID NO: 2; SEQ ID NO: 3; SEQ ID NO: 4 or a or a Coronavirus/Coronavirus antigen binding fragment thereof;
(iv) an antibody which binds the spike (S)-protein, the S1 domain of the S-protein, the S2 domain of the S-protein and/or the receptor binding domain (RBD) of the S-protein.
(v) an antibody which binds the same target as the first binding agent; and
(vi) an antibody which binds the S2 domain of the S-protein.

44. The method of claim 42, wherein the second binding agent is selected from the group consisting of:
(i) ACE2 or a Coronavirus/Coronavirus antigen binding fragment thereof; and
(ii) a protein having a sequence of SEQ ID NO: 1, SEQ ID NO: 2 or a Coronavirus/Coronavirus antigen binding fragment thereof.
